(19) United States
(12) Patent Application Publication
(10) Pub. No.: US 2004/0110177 A1
(43) Pub. Date: Jun. 10, 2004

(54) Method for identifying functional nucleic acids

(76) Inventors: Axel Ullrich, Munchen (DE); Reimar Abraham, Munchen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/470,845

(22) PCT Filed: Feb. 1, 2002
(86) PCT No.: PCT/EP02/01073

(51) Int. Cl. C12Q 1/68

(52) U.S. Cl. 435/6

(57) ABSTRACT

The present invention relates to a method for identifying nucleic acid molecules functionally associated with a desired phenotype.

The present invention relates to a method for identifying nucleic acid molecules functionally associated with a desired phenotype.

A lot of information has been gathered about the execution apparatus of apoptosis (Hengartner, Nature 407 (2000), 770-776). But data on signals that control the initiation of apoptosis have only recently begun to be accumulated (Rich et al., Nature 407 (2000), 777-783). Previous methods for identifying apoptosis-associated genes or genes associated with other specific phenotypes are tedious. For example, Hudziak et al. (Cell Growth and Differentiaton 129 (1990), 129-134) describe a selection procedure for transformation and met protoonco gene amplification in NIH 3T3 fibroblasts using tumor necrosis factor-α. It is suggested that this method may be used for identifying other gene products, including other tyrosine kinases, associated with aggressive tumor growth. A fast or reliable procedure for identifying such genes is, however, not provided.

According to the present invention a novel method for identifying functional nucleic acid molecules is provided. This method is based on a genome evolution concept and therefore involves mutagenesis and/or genome arrangement steps followed by selection of cell clones displaying the desired phenotype. Subsequent transcriptome analysis in conjunction with bioinformatics-directed gene sorting allows not only comprehensive identification of genes that are critical for the selected cell characteristic, but even entire signalling pathways that govern a given cellular phenotype. This method can be employed towards a wide variety of cell characteristics for which a selection procedure is available.

Thus, a subject matter of the present invention is a method for identifying nucleic acid molecules functionally associated with a desired phenotype comprising the steps:

(a) providing a population of parental cells wherein said cell population substantially lacks the desired phenotype,

(b) optionally subjecting said cell population to a procedure resulting in a rearrangement and/or mutation of the cell genome,

(c) subjecting said cell population from (b) to a selection procedure for the desired phenotype,

(d) identifying and optionally characterizing cells exhibiting said desired phenotype,

(e) obtaining protein and/or mRNA from cells exhibiting said desired phenotype,

(f) determining gene expression in cells exhibiting said desired phenotype and

(g) comparing gene expression in cells exhibiting said desired phenotype with gene expression in cells substantially lacking the desired phenotype.

In the method of the invention essentially any type of parental cells (e.g. cell lines or primary cells) can be used. Most important the cells should lack the desired selection characteristic or display it only weakly. Preferred examples of starting cells are eukaryotic cells, e.g. mammalian cells, particularly human cells.

In order to generate cells, preferably cell clones exhibiting the desired phenotype, the parental cell may be subjected to a procedure resulting in an arrangement and/or mutation of the cell genome. This step is an evolution procedure comprising an induction of the parental cell to undergo genomic rearrangements and/or mutagenesis. In case of transformed cells, e.g. tumor cells such as Hela or normal cells having a low threshold to instability, e.g. immortalized cells such as NIH 3T3 cells, no special induction is necessary, since these cells are continuously in a process of genome rearrangement and mutagenesis. It is sufficient to expose the parental cell culture to selection conditions either in form of clones or subdivided cultures preferably in multiple well plates, e.g. 96 well microtiter plates, or when the selection involves lethal conditions, exposure of cell monolayers. It should be noted, however, that also parental cells may be used which have a substantially stable genome. These cells, however, require a specific induction in order to obtain the desired genomic rearragement and/or mutagenesis.

In a preferred embodiment step (b) of the method comprises a mutagenesis procedure. This mutagenesis procedure may be selected from irradiation, e.g. by UV or γ-irradiation, chemical mutagenesis, e.g. by treatment with N-methyl maleimide or ethyl maleimide, or combinations thereof.

After the rearrangement and/or mutation of the cell genome has been achieved, the cell population is subjected to a selection procedure for the desired phenotype. After selection, cells, e.g. individual cell clones exhibiting the desired phenotype are identified and optionally characterized. The identification may comprise a morphological determination and/or a cell sorting procedure, e.g. by a Fluorescence Activated Cell Sorting procedure (FACS). The cells may be expanded and subsequently the desired phenotype/property may be verified and/or quantified.

Subsequently, protein and/or mRNA from cells exhibiting the desired phenotype is obtained. This material may be used for determining gene expression in cells exhibiting the desired phenotype and comparing gene expression in said cells with gene expression in cells substantially lacking the desired phenotype.

In a preferred embodiment, mRNA from cells exhibiting the desired phenotype is obtained. The mRNA may be extracted from the selected genetically modified cell clones and either used directly, or after conversion into another nucleic acid, e.g. cDNA or cRNA as a probe for hybridization with a nucleic acid array. The nucleic acid, e.g. mRNA, cDNA or cRNA, used for hybridization with the array will usually be labelled in order to determine site-specific hybridization on the array. The array may be a solid carrier, e.g. a filter, chip, slide etc. having immobilized thereto a plurality of different nucleic acid molecules on specified locations on the carrier. The nucleic acid array may be selected from genomic DNA arrays, cDNA arrays and oligonucleotide arrays. Preferably, an array is used which preferentially comprises nucleic acids encoding functional cellular polypeptides or portions thereof, more preferably selected from kinases, phosphatases, enzymes and receptors. Hybridization on the array as a measure of gene expression in the selected cell clones may be determined according to known methods, e.g. by image analyis using a phosphor imager. In some cases, the desired new property of the cell may be determined by a large scale high throughput assay analysis of e.g. the conditioned media of subdivided cultures.

In addition or alternatively to expression profiling by mRNA analysis a proteomics approach determining the differences in protein content of the identified clones compared to the parental cell line and the identified clones or their supernatants may be carried out by suitable methods, e.g. by 2D gel electrophoresis. Proteins that differ in their concentration in the parental cell line and the identified clones will show a differently stained spot in the 2D gel. Furthermore, protein modifications like phosphorylations can be detected by this method. Once can also perform a separation of the cellular proteins prior to the analysis step, in order to reduce the complexity of the protein mixture. For instance, column chromatographic steps could be carried out that purify kinases (by affinity chromatography using an ATP column) or glycosylated proteins (using a lectin column) which then can be further separated by 2D gel electrophoresis. Any other method for analyzing differences on the protein level (protein chips, mass spectrometry) may also be utilized.

The gene expression results in cells exhibiting the desired phenotype will be compared with gene expression in cells substantially lacking the desired phenotype, preferably in the parental cells. Further, the gene expression results may be analyzed by a cluster detection program. This analysis will yield a plurality of possible changes in the expression of genes that confer the desired cell phenotype.

The application of the method of the invention is very broad and includes essentially all cell characteristics that can be selected for and/or which can be determined with an assay. For example, the desired phenotype may be selected from cancer cell properties such as invasiveness, metastasis, loss of contact inhibition, loss of extracellular matrix requirement, growth factor independence, angiogenesis induction, immuno defense evasion, anti-apoptosis and/or increased levels of tumor markers.

In an especially preferred embodiment the desired phenotype is anti-apoptosis. Another application is the elucidation of cancer related genes by sorting cancer cells for a known tumor marker. Often tumor markers are a consequence and not a cause of the tumorigenicity of cells and are therefore not amenable as drug targets. But since the correlation of the marker with a cancer phenotype is established, sorting cells for increased marker expression will also sort for the genes that are linked to the marker and cause the cancer phenotype. These genes can be identified by comparing the expression profiles in the parental cell line and the sorted cells and are potential drug targets.

Alternatively, the desired phenotype may be selected from other properties such as production of secreted protein, e.g. insulin, growth hormone, interferons etc., susceptibility or resistance to pathogens, e.g. viruses such as HCV, HBV or other pathogens, senescence and regulation of cell functions, i.e. the identification of genes that regulate certain cell functions e.g. identification of negative regulators of insulin receptor activity comprising a screen for cell clones with upregulated insulin receptor activity.

A further preferred embodiment is the identification of components of signal transduction pathways in general, e.g. to sort for cells that are better capable of transmitting the respective signal. For instance, the identification of components of a signal transduction pathway of a Receptor Tyrosine Kinase (RTK), particularly of a receptor of the EGF-receptor family, such as EGFR, HER2 and HER3, can be carried out by generating a cell line that expresses a suitable reporter protein, such as Green Fluorescent Protein (GFP) under the control of a promoter that is responsive to stimulation by a ligand of the respective receptor (e.g. c-fos promoter for EGF stimulation etc.). Stimulation of the receptor by the ligand will then lead to transcription of GFP and an increased green fluorescence that can be detected, e.g. by a FACS machine. Sorting the cells that show the highest fluorescence induction will enrich for cells that respond stronger to a ligand-indicated signal than the parental cell population. Analyzing the expression patterns of both cell populations will identify the genes whose varying expressions are responsible for the different reaction to the signal and hence influence the signal transduction pathway. This strategy can be applied to any signal for which a fluorescent output can be generated.

In the following, the invention is described in more detail with reference to the identification of anti-apoptotic nucleic acids using a cDNA array. It should be noted, however, that this embodiment is only illustrative for the method of the invention and should not be construed as limitation.

In order to identify nucleic acids which are associated with the regulation of apoptosis the method of the invention was used for the identification of genes, which are differentially expressed in apoptosis-sensitive and apoptosis-resistant cells.

Apoptosis was induced in the human cervix carcinoma cell line Hela S3 by Fas activation. Activation of Fas results in an autocatalytic activation of caspase-8 and thus to apoptosis. For Fas activation the parental cells were incubated with an anti-Fas antibody.

After the selection procedure only a low amount of living cells were present. These cells had a higher resistance against apoptosis than the parental cell line. The surviving cells were clonally expanded. mRNA was isolated from the clones and the parental cell line, which was subsequently reversed, transcribed into cDNA. Then cDNA arrays were hybridized with the cDNA from the clones and the parental cell line and thus the gene expression on the array determined. The sequences on the arrays were derived from about 1000 genes which preferentially encode kinases and phosphatases. By means of a comparison between the expression and the parental cell line and the expression and the clones, about 200 genes were identified which exhibited enhanced expression (an increase by more than the factor 2) in at least 10% of the clones. These are nucleic acids which are associated with the apoptosis resistance of the clones (Tables 1 and 2). Table 1 is a listing of genes which are induced in the apoptosis-resistant clones and have not yet been linked to an anti-apoptosis function. Table 2 is a listing of genes that are induced in apoptosis-resistant clones with previously known anti-apoptotic function.

An improved method for the identification of genes, which are differentially expressed in the parental cell line, e.g. Hela S3, and the clones having a desired phenotype, e.g. apoptosis-resistant clones, an evaluation procedure as described in Example 2, may be applied. For each nucleic acid analysed in the parental cell line, a plurality of measured values is determined from which an average value and a standard deviation may be calculated. For example, RNA may be isolated at least twice from the parental cell line in at least two independent preparations. Material from each preparation is used for hybridization with at least two nucleic acid arrays. The average of those values for a given spot on the array is calculated and the standard deviation determined. Material from the desired clone is hybridized with one nucleic acid array. A gene is considered to be differentially expressed in the desired clone when its value exceeds a predetermined cut-off. The cut-off for upregulated genes is preferably the average of the respective values of the parental cell line plus two times standard deviation. The cut-off for down-regulated genes is preferably the average of the respective parental cell line values minus two times standard deviations. Using this procedure it is possible to correct errors inherent in the experimental procedure. Since those errors made during the preparation of the nucleic acid arrays will determine the standard deviation, any value of the desired clone that lies outside the standard deviation marks a differentially expressed gene. Therefore, it is possible to detect also small differences in gene expression that may not be detected by using an arbitrary cut-off. The values obtained by this improved evaluation procedure are depicted in Table 5.

Thus, a subject matter of the present invention is the use of nucleic acids as depicted in Table 1, Table 2, and Table 5 preferably in Table 1 and Table 5, and polypeptides encoded by these nucleic acids as “targets” for diagnostic and therapeutic applications, particularly for disorders which are associated with dysfunctions of apoptotic processes such as tumors. Further, the nucleic acids and the gene products are suitable as targets in screening procedures for identifying novel modulators of apoptotic/anti-apoptotic procedures, particularly drugs. The drugs may be biomolecules such as antibodies directed against the gene products, enzyme inhibitors or low molecular non-biological drugs. Methods of drug screening comprise cellular based systems wherein usually a cell overexpressing the target nucleic acid of interest is used or molecular based systems wherein the polypeptide of interest in used in a partially purified or substantially purified and isolated form. Particular screening methods are known to the skilled person and need not be described in detail here. It should be noted, however, that also high throughput screening assays may be used.

Further, several groups or clusters of genes were identified whose expression patterns across the cell lines are similar. Clusters of apoptosis-resistant clones are depicted in Table 3. Clusters in squamous cell carcinoma cell lines are depicted in Table 4. The identification of such clusters allows the use of specific combinations of active agents in diagnostic and/or therapeutical applications as well as in screening methods. Thus, according to a preferred embodiment of the invention combinations of agents capable of modulating the presence and/or activity of several targets within a cluster may be used in order to multiply the efficacy.

Furthermore, the method of the present invention allows the generation of expression profiles of genes and particularly gene clusters associated with a desired phenotype. These expression profiles may be compared with the expression profile in a specific biological sample, which may be a body fluid or a tissue sample derived from a patient, e.g. a human, particularly a tumor patient. The comparison of the expression profile obtained by the method of the present invention with the expression profile in the biological samples allows the development of improved diagnostic, monitoring and/or therapeutic strategies which are specifically adapted to the individual patient.

In experiments it was demonstrated that an inhibition of the catalytic activity of proteins having an increased expression in the clones resulted in an enhanced increase of apoptosis. Also in the parental cell line the inhibition resulted in an increased apoptosis. This outlines the importance of the identified nucleic acids and proteins for the apoptosis resistance of the clones and demonstrates the inhibition specifity.

Further, the invention is described in more detail in the following examples and figures.

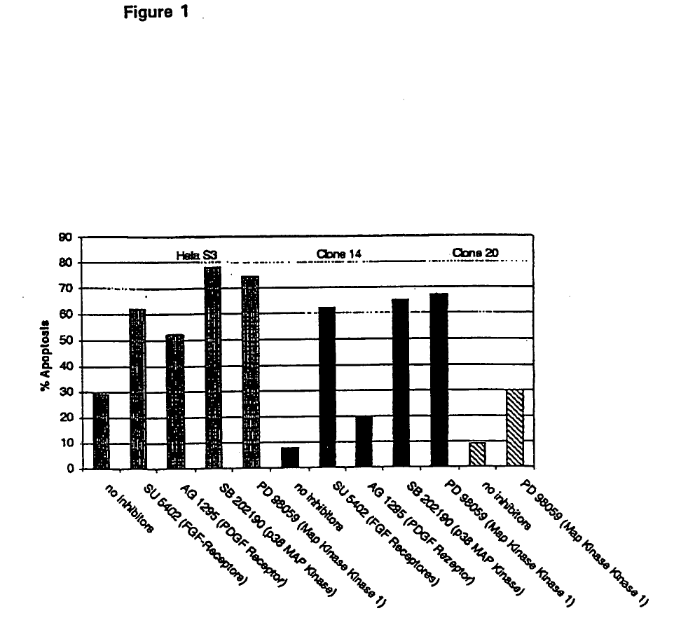

FIG. 1 shows the inhibition of upregulated kinases.

Cells were grown in Ham's F12 medium without FCS and treated with 100 ng/ml anti-Fas antibody CH-11 with and without inhibitors. Apoptosis was measured by FACS analysis as described in the examples. SU 5402: 10 μM, AG 1295: 1 μM, SB 203580: 10 μM, PD 98059: 25 μM.

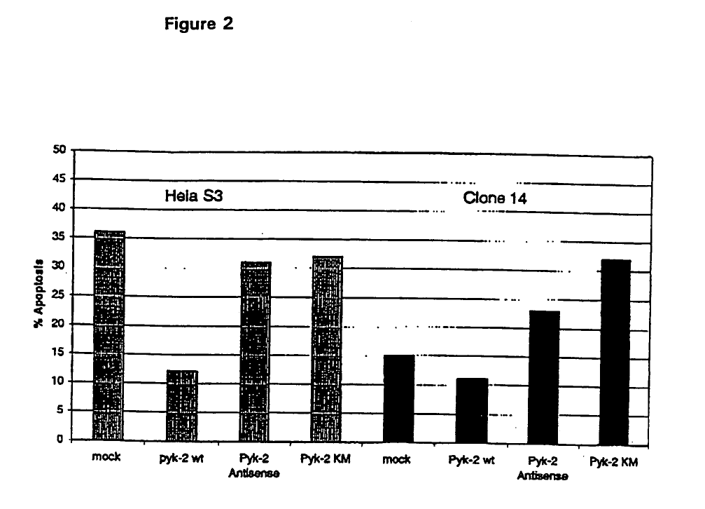

FIG. 2 shows the inhibition of pyk-2 by a dominant negative mutant and an antisense construct.

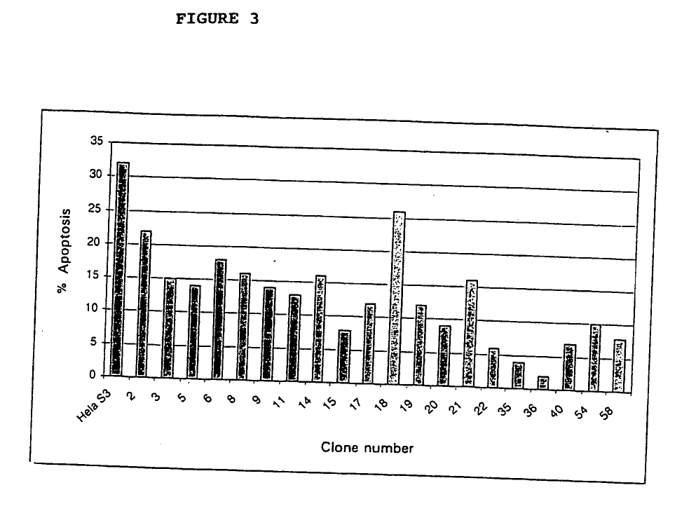

FIG. 3 shows the apoptosis sensitivity of clones. 70% confluent cells were starved for 24 h in medium without FCS and subsequently 100 ng/ml CH-11 was added. After a 16 h incubation the cell nuclei were stained in hypotonic buffer and analysed by FACS. The percentage of the sub-G1-peak was deduced. The apoptotic rate without FCS was subtracted from the rate with FCS.

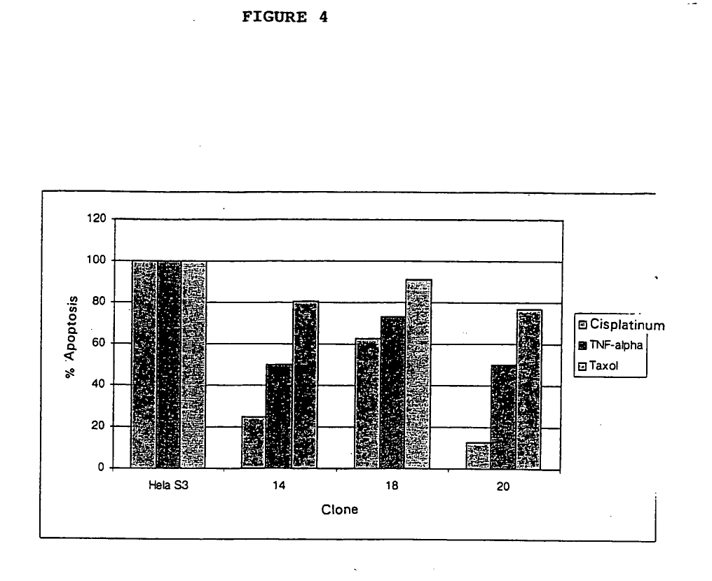

FIG. 4 shows the apoptosis sensitivity with other apoptosis inducers. 70% confluent cells were starved for 24 h in medium without FCS and subsequently 10 μg/ml Cisplatinum or TNF-α plus 0.1 μg/ml Cycloheximide was added to the cells. After 16 h the cell nuclei were stained with propidium iodide and analysed by FACS. 50 nM Taxol was added to the cells for 3 h and the medium subsequently replaced by fresh medium with 10% FCS. 2 days later the percentage of sub-G1 cells was deduced. The apoptotic rate without FCS was subtracted from the rate with FCS. The values are expressed as the percentage of the respective Hela S3 value.

Viral supernatant was produced using Phoenix A packaging cell line and the respective cloned constructs (expressing pyk-2 wild-type or pyk-2 KM mutant) cloned in the vector pLXSN. Hela S3 and clone 14 were infected over night. Medium was changed the next day and two days later cells were starved for 24 hours in medium without FCS before adding 100 ng/ml CH-11 over night. Apoptosis was measured as described in FIG. 1.

### EXAMPLE 1

1. Materials and Methods

1.1 Selection of Apoptosis-Resistant Clones

The cervix carcinoma cell line Hela S3 (ATCC CCL-2.2) was plated on 10 cm cell culture dishes (10⁵ cells) in Ham's F12 growth medium containing 10% FCS. On the next day the medium was exchanged against medium without FCS supplemented with 100 ng/ml apoptosis activating anti-Fas antibody CH-11 (Coulter Immunotech). After 3 days when most of the cells were dead, the medium was exchanged once more against the medium containing 10% FCS without antibody. The surviving cells were clonally cultivated for 3 weeks. The clones were picked and expanded.

1.2 Apoptosis Assay

50000 cells per well obtained from the parental cell line Hela S3 or from the clones, respectively, were grown in a 12 well cell culture dish for 2 days in 2 ml Ham's F12 medium containing 10% FCS. On the third day the cells were washed twice with 1 ml Ham's F12 medium and then the medium exchanged against 1 ml Ham's F12 medium. On the next day the medium was supplemented with the respective inhibitors and 100 to 200 ng/ml CH-11. On the next day the medium was decanted and transferred to an Eppendorf tube. The cells were washed once with 200 μl PBS, the PBS was transferred to the respective Eppendorf tube. Then the remaining cells were also transferred to the respective Eppendorf tube after treatment with EDTA/trypsin in PBS. The cells were pelleted by centrifugation, suspended in 500 μl hypotonic buffer (0.1% sodium citrate, 0.1% Triton-X100, 20 μg/ml propidium iodide) and incubated for 2-24 hours at 4° C. The resutling cell nuclei were analyzed by FACS.

1.3 FACS (Fluorescence Activated Cell Sorting)—Analysis for Determining Apoptotic Nuclei

The propidium iodide fluorescence of single nuclei was determined using a FACSCalibur (Becton Dickinson) cytometer. The forward scatter light (FSC) and the side scatter light (SSC) were recorded simultaneously. The FSC peak was adjusted at channel 500 in a 1024 channel linear scale and the red fluorescence peak at channel 200 of a logarithmic scale. The FSC cut-off value was determined by gating to 95% of the greatest nuclei of a negative control without supplements. Nuclei were classified as apoptotic when a subdiploid signal between the G1/G0 peak and channel 10 was present.

1.4 Preparation of cDNA

Total RNA was isolated by lysing of cells with guanidinium isothiocyanate and subsequent extraction with acid phenol (Current Protocols in Molecular Biology). mRNA was isolated by binding to oligo-dT cellulose according to standard methods (Current Protocols in Molecular Biology).

cDNA was synthesized from mRNA by reverse transcription using Cap-finder primer K1 and K2 (Clontech Inc., USA) and AMV-reverse transcriptase (Roche Diagnostics) and purified using the PCR purification kit (Qiagen). From 3 μg mRNA 50 μl cDNA consisting of one strand DNA and one strand RNA were obtained.

1.5 Preparation of cDNA Arrays

cDNAs cloned in p-Blu script were spotted with a BioGrid spotter (BioRobotics, UK) on nylon membranes. 250 ng DNA were used per spot. For about one half of the genes two or more probes were used and each probe was spotted twice. The following designations were used:

YK=tyrosine kinase

STK=serin/threonin kinase

PP=phosphatase

Lig=ligand

UK=unknown kinase

UP=unknown phosphatase

OT=other

Example:

YK_{—}1b_Abl_{—}2=tyrosine kinase 1, probe b, spot 2

1.6 Radioactive Labelling of cDNA

5 μl cDNA were labelled with 50μ Ci α³³P-ATP using the Megaprime Labelling Kit (Amersham Pharmacia) and purified using the PCR purification kit (Qiagen). The thus obtained cDNA was hybridized with COT-DNA (Roche Diagnostics) in order to block repetitive sequences which might bind unspecifically to the cDNA array.

1.7 Hybridization of cDNA Arrays

The cDNA arrays were prehybridized for 4 hours or over night at 68° C. in prehybridization solution (50×Denhardt, 10×SSC, 0.25 M Na₃PO₄, pH 6.8, 50 mM Na₄P₂O₇, 0.1 mg/ml tRNA (bakers's yeast, Roche Diagnostics)).

Subsequently the cDNA arrays were hybridized for 16 hours with the labelled cDNA in hybridization buffer (5×SSC, 0.1% SDS, 0.1 mg/ml tRNA). The cDNA arrays were washed as follows:

2×20 min W1 (2×SSC, 0.1% SDS) at 42° C.

1×20 min W2 (0.2×SSC, 0.1% SDS) at 42° C.

1×60 min W2 at 65° C.

The cDNA arrays were exposed for 48 hours on Phosphoimager plates (Fujifilm) and subsequently analyzed on a Phosphoimager (Bas-2500, Fujifilm).

1.8 Analysis of cDNA Arrays

The spot volume on the filter was determined using ArrayVision software (V 5.1, Imaging Research Inc.). All further calculations were carried out in Excel (Microsoft Corp.).

For better internal comparison of the cDNA arrays a normalization procedure was carried out as follows: From each spot on the array the background (average of p-Bluescript values of an array) was subtracted and divided by the sum of all spot volumina in the array. The thus obtained value was multiplied by 10000.

For the identification of genes which are differentially expressed in the parental cell line Hela S3 and the apoptosis-resistant clones, the quotient from the values of the clones and the average value of the different arrays of the parental cell line (reference arrays) was calculated. All normalized values smaller than 0.1 were set to 0.1 for the calculation. 90% of all values different from 0 were above this value. The respective gene was defined as differentially expressed, if the percentage differs by at least 100%. Only such genes were analyzed wherein the deviation of the values on the reference arrays for the respective spot on the array was sufficiently small. The following filters were used for sorting out these genes:

If the values of the reference arrays and of the respective clone for a spot were smaller than 2.5, the deviation of the reference arrays from each other must be in the range from 0.2 to 5.

If the values of the reference arrays were smaller than 2.5 and that of the clone greater than 2.5 or vice versa, the deviation of the reference arrays from each other has to be in the range from 0.3 to 3.

If both the values of the reference arrays and of the clone were greater than 2.5, the deviation of the reference arrays from each other has to be in the range from 0.5 to 2.

1.9 Gene Clustering

For gene clustering the Program Cluster (Michael Eisen, Stanford University) was used. The quotients from the values of the clones and the average value of the respective arrays of the parental cell lines were used. Spots exhibiting high deviations in the values on the reference arrays were excluded. For this purpose the filters were used which had already been applied in the identification of induced genes. From 1922 spots 1451 remained. These values were logarithmically transferred to clusters and further filtered on spots wherein the value of at least 80% of the clones was different from 0. The thus resulting 520 spots were analyzed via an hierarchical cluster algorithm.

The overall similarity of the expression patterns and the cluster mirrors in the correlation coefficient which has a value between 1 and −1. A correlation coefficient of 1 means the expression patterns are identical, 0 means that they are completely independent and −1 the opposite of each other.

2. Results

2.1 Apoptosis-Resistant Clones are Obtained by Selection of Hela S3 Using CH-11 Antibody

40 clones were obtained after selection with CH-11 antibody. 20 of these clones were tested in view of their sensitivity to CH-11. The degree to which the clones are resistant differs between individual clones, but none of them is completely resistant to apoptosis suggesting that the apoptosis machinery is functional. The clones are also refractive to apoptosis induced by TNF-α and cisplatin.

2.2 Numerous Genes Show Enhanced Expression in Apoptosis-Resistant Clones

Tables 1 and 2 show listings of genes which show enhanced expression in apoptosis-resistant clones. Further, the Genbank Accession numbers of the respective clones, the number of clones in which expression exceeds cut-off for increased expression and the average percentage over cut-off is given.

Most of the analyzed genes encode protein phosphatases and kinases, i.e. enzymes which are important for cell regulation.

From the thus determined induced clones several have not yet been associated with apoptosis and/or tumorogenesis (Table 1). Other genes such as CAMKK (calmodulin dependent kinase kinase), EGFR (epidermal growth factor receptor), Bcr (breakpoint cluster region), FGFR-1 (fibroblast growth factor receptor 1), Nik (NFκB-interacting kinase) and DAPK (death-associated protein kinase) are already known as apoptosis-associated genes.

2.3 Gene Clustering Shows Groups of Genes Which are Commonly Regulated

By clustering of expression data groups of genes were found which are commonly up- or downregulated. The common regulation suggests a common function of the genes. Thus not only single apoptosis-modulating genes, but also signal transduction cascades consisting of a plurality of genes are found. The clusters identified in apoptosis-resistant clones are shown in Table 3. The clustering of the genes allows to group the upregulated genes and deduce different anti-apoptotic signalling pathways instead of single genes only. The clusters that were found in the apoptosis-resistant clones could also be partially found in expression data of squamous cell carcinoma cell lines (Table 4). That suggests that by the screen physiologically relevant apoptosis clusters can be found that are important for tumor development and hence could serve as drug targets.

Cluster 1 contains some genes induced in many clones such as CAMKK, UK11 (unknown kinase 11), PTP α (protein tyrosine phosphatase α) and PRK (proliferation related kinase).

Cluster 2 contains 3 genes exhibiting a highly correlated expression, namely serin/threonin phosphatase VH2, TIMP (tissue inhibitor of metalloproteinase 1) and MMP-15 (matrix metalloproteinase 15). Interestingly, an enzyme (MMP-15) and a potential inhibitor (TIMP-1) are commonly regulated.

Cluster 3 comprises inter alia the membrane bound tyrosine phosphatase Lar and the proapoptotic serin/theronin kinase DAP kinase.

In cluster 4 BCR, a potential inhibitor of p38 and the JNK signal pathways, and an activator of p38, namely MAPKK-3 (mitogen activated kinase kinase 3) are commonly regulated.

2.4 Inhibition of the Induced Genes Enhances Apoptosis

In order to show that th induced genes are in fact modulators of apoptosis selected enzymes were inhibited by specific inhibitors and apoptosis was induced. Inhibitors for the following enzymes were used:

SU 5402 inhibits FGF receptors, but is not specific for a defined FGF receptor

AG 1295 inhibits the PDGF receptor

SB 203580 inhibits the p38 MAP kinase

PD 98059 inhibits the MAP kinase kinase 1, which in turn activates the MAP kinases ERK1 and ERK2. This inhibitor was used as control for SB 203580, because SB 203580 also partially inhibits ERK1 and ERK2. Furthermore, ERK2 shows an enhanced expression in the clones. The results for Hela S3, clone 14 and clone 20 (partially) are shown in FIG. 1.

It was found that an inhibition of FGF receptors in Hela S3 cells leads to an increase in apoptosis of about 50%. In clones 14 and 5 SU 5402 leads to an increase of nearly 300% or 50%, respectively. Thus, in a clone having an increased expression of two FGF receptors (clone 14, FGFR-1 and FGFR-3) an inhibition of FGF receptors leads to an enhanced increase of apoptosis. In clone 5, which does not show any enhanced expression of FGF receptors, the increase in apoptosis is comparable to the parental cell line Hela S3.

An inhibition of the PDGF receptor leads to an increase of about 30% in Hela S3. In clone 14, which shows enhanced expression of PDGF receptor, the inhibition results nearly in a doubling of the number of apoptotic cells. In contrast thereto, clone 5, which does not contain any detectable PDGF receptor, exhibits only 30% increase in apoptosis after treatment with AG 1295.

The p38 MAP kinase was inhibited because BCR, an inhibitor of the p38 MAP kinase signal pathway, and MAPKK-3 (MEK-3), which is a p38 activator, exhibited an enhanced expression in the clones. Further, both genes are grouped in a cluster.

p38 inhibition in Hela S3 results in a 25% increase of apoptosis. In clone 14 exhibiting an enhanced MEK-3 expression, an inhibition of p38 leads to a 60% increase of apoptosis. In contrast thereto, an inhibition of MEK-1 results in a doubling of the apoptosis rate. The increase in apoptosis after inhibition of p38 compared to Hela S3 and the constant apoptosis after inhibition of MEK-1 might be explained by inhibition of ERK1/2 and additional inhibition of p38.

In clone 20, which expresses MEK-3 on a similar level as Hela S3, treatment with SB 203580 only leads to a slight increase of apoptosis. In contrast thereto, treatment with PD 98059 triples the apoptosis rate. Thus, SB 203580 acts specifically in this system and the differences in the increase of apoptosis after inhibition of p38 correlate with the expression of the p38 activator MEK-3.

These inhibition experiments demonstrate conclusively that the method of the invention for identifying apoptosis-associated genes is efficient.

2.5 Inhibition by Introducing a Dominant Negative Mutant or an Antisense Strand

The respective enzymes upregulated in apoptosis-resistant clones can also be inhibited by introducing a dominant negative mutant or the antisense strand. FIG. 2 shows that—as as example—the wild-type pyk-2 confers increased resistance when introduced in Hela S3. In clone 14 with a higher expression of pyk-2 introduction of the wild-type enzyme has no effect but the mutant with the lysine mutated to methionine (pyk-2 KM) in the reactive center of the enzyme reverts the phenotype of the clone. The antisense construct has a corresponding but weaker effect.

### EXAMPLE 2

The experimental procedure was carried out as described in Example 1.

For the identification of genes differentially expressed in the parental cell line Hela S3 and the apoptosis resistant clones, the following evaluation procedure was applied. For each spot on the cDNA arrays of the parental cell line Hela S3 four values were determined in the following manner. RNA was isolated twice from Hela S3 in two independent preparations. Each RNA preparation was used to synthesize cDNA and each cDNA was hybridized with two cDNA arrays. The average of those 4 values for a given spot on the cDNA array was calculated and the standard deviation determined. The cDNA of each apoptosis resistant clone was hybridized with one cDNA array. A gene was considered to be differentially expressed in the apoptosis resistant clones when its value exceeded the following cut offs. The cut off for upregulated genes was the average of the respective Hela S3 values plus two times standard deviation. Accordingly, the cut off for downregulated genes was the average of the respective Hela S3 values minus two times standard deviation. The magnitude of the up- or downregulation was expressed as percent over/under the cut off. For example, a value of 100% over the cut off for upregulated genes means a 2-fold induction compared to the cut off, and a value of 100% under the cut off for downregulated genes means a bisection of that value in the resistant clones.

For gene clustering the program Cluster (Michael Eisen, Stanford University) may be used. The normalized values of the four reference arrays and the array of the 20 apoptosis resistant clones were used. Genes with a value greater than 1 in at least 20 of the 24 investigated arrays were filtered out and employed for the following calculations. The cut off For gene clustering the program Cluster (Michael Eisen, Stanford University) may be used. The normalized values of the four reference arrays and the array of the 20 apoptosis resistant clones were used. Genes with a value greater than 1 in at least 20 of the 24 investigated arrays were filtered out and employed for the following calculations. The cut off of 1 was utilized in order to avoid clustering of genes whose value was so close to the background that a clustering would be unreliable. Thus, out of 2400 spots, 520 remained that were analysed via a hierarchical cluster algorithm.

The results are shown in Table 5.

References:

1. Will, H. and B. Hinzmann, cDNA sequence and mRNA tissue distribution of a novel human matrix metalloproteinase with a potential transmembrane segment. Eur J Biochem, 1995. 231(3): p. 602-8.

2. Butler, G. S., et al., Membrane-type-2 matrix metalloproteinase can initiate the processing of progelatinase A and is regulated by the tissue inhibitors of metalloproteinases. Eur J Biochem, 1997. 244(2): p. 653-7.

3. Hu, M. C., et al., Protein phosphatase X interacts with c-Rel and stimulates c-Rel/nuclear factor kappaB activity. J Biol Chem, 1998. 273(50): p. 33561-5.

4. Kersh, G. J. and P. M. Allen, Essential flexibility in the T-cell recognition of antigen. Nature, 1996. 380(6574): p. 495-8.

5. Kohno, K., et al., Amino acid sequence of mammalian elongation factor 2 deduced from the cDNA sequence: homology with GTP-binding proteins. Proc Natl Acad Sci U S A, 1986. 83(14): p. 4978-82.

6. Geiger, B., Intermediate filaments. Looking for a function. Nature, 1987. 329(6138): p. 392-3.

7. Didsbury, J. R., Y. S. Ho, and R. Snyderman, Human Gi protein alpha-subunit: deduction of amino acid structure from a cloned cDNA. FEBS Lett, 1987. 211(2): p. 160-4.

8. Bokoch, G. M. and A. G. Gilman, Inhibition of receptor-mediated release of arachidonic acid by pertussis toxin. Cell, 1984. 39(2 Pt 1): p. 301-8.

9. Sullivan, K. A., et al., Inhibitory and stimulatory G proteins of adenylate cyclase: cDNA and amino acid sequences of the alpha chains. Proc Natl Acad Sci U S A, 1986. 83(18): p. 6687-91.

10. Williams, N. G., H. Zhong, and K. P. Minneman, Differential coupling of alpha1-, alpha2-, and beta-adrenergic receptors to nitrogen-activated protein kinase pathways and differentiation in transfected PC12 cells. J Biol Chem, 1998. 273(38): p. 24624-32.

11. Mukai, H. and Y. Ono, A novel protein kinase with leucine zipper-like sequences: its catalytic domain is highly homologous to that of protein kinase C. Biochem Biophys Res Commun, 1994. 199(2): p. 897-904.

12. Wick, M. J., et al., Mechanism of phosphorylation of protein kinase B/Akt by a constitutively active 3-phosphoinositide-dependent protein Kinase-I. J Biol Chem, 2000. 275(51): p. 40400-6.

13. Takahashi, M., et al., Proteolytic activation of PKN by caspase-3 or related protease during apoptosis. Proc Natl Acad Sci U S A, 1998. 95(20): p. 11566-71.

14. Watanabe, G., et al., Protein kinase N (PKN) and PKN-related protein rhophilin as targets of small GTPase Rho. Science, 1996. 271(5249): p. 645-8.

15. Mukai, H., et al., Activation of PKN, a novel 120-kDa protein kinase with leucine zipper-like sequences, by unsaturated fatty acids and by limited proteolysis. Biochem Biophys Res Commun, 1994. 204(1): p. 348-56.

16. Maru, Y. and O. N. Witte, The BCR gene encodes a novel serine/threonine kinase activity within a single exon. Cell, 1991. 67(3): p. 459-68.

17. Diekmann, D., et al., Bcr encodes a GTPase-activating protein for p21rac. Nature, 1991. 351(6325): p. 400-2.

18. Galaktionov, K. and D. Beach, Specific activation of cdc25 tyrosine phosphatases by B-type cyclins: evidence for multiple roles of mitotic cyclins. Cell, 1991. 67(6): p. 1181-94.

19. Honda, R., et al., Dephosphorylation of human p34cdc2 kinase on both Thr-14 and Tyr-15 by human cdc25B phosphatase. FEBS Lett, 1993. 318(3): p. 331-4.

20. Sebastian, B., A. Kakizuka, and T. Hunter, Cdc25M2 activation of cyclin-dependent kinases by dephosphorylation of threonine-14 and tyrosine-15. Proc Natl Acad Sci U S A, 1993. 90(8): p. 3521-4.

21. Miyata, H., et al., Overexpression of CDC25B overrides radiation-induced G2-M arrest and results in increased apoptosis in esophageal cancel cells. Cancer Res, 2001. 61(7): p. 3188-93.

22. Zuo, Z., et al., Ser/Thr. protein phosphatase type 5 (PP5) is a negative regulator of glucocorticoid receptor-mediated growth arrest. Biochemistry, 1999. 38(28): p. 8849-57.

23. Silverstein, A. M., et al., Protein phosphatase 5 is a major component of glucocorticoid receptor hsp90 complexes with properties of an FK506-binding immunophilin. J Biol Chem, 1997. 272(26): p. 16224-30.

24. Chen, M. X., et al., A novel human protein serine/threonine phosphatase, which possesses four tetratricopeptide repeat motifs and localizes to the nucleus. Embo J, 1994. 13(18): p. 4278-90.

25. Lumeng, C., et al., Interactions between beta 2-synthrophin and a famiiy of microtubule-associated serine/threonine kinases. Nat Neurosci, 1999. 2(7): p. 611-7.

26. Walden, P. D. and N. J. Cowan, A novel 205-kilodalton testis-specific serine/threonine protein kinase associated with microtubules of the spermatid manchette. Mol Cell Biol, 1993. 13(12): p. 7625-35.

27. Yoshiyama, K., et al., CD156 (human ADAM8): expression, primary amino acid sequence, and gene location. Genomics, 1997. 41(1): p. 56-62.

28. Schlomann, U., et al., Tumor necrosis factor alpha induces a metalloprotease-disintegrin, ADAM8 (CD 156): implications for neuron-glia interactions during neurodegeneration. J Neurosci, 2000. 20(21): p. 7964-71.

29. Wang, Z., R. Jutternann, and P. D. Soloway, TIMP-2 is required for efficient activation of proMMP-2 in vivo. J Biol Chem, 2000. 275(34): p. 26411-5.

30. Zucker, S., et al., Tissue inhibitor of metalloproteitiase-2 (TIMP-2) binds to the catalytic domain of the cell surface receptor, membrane type 1-matrix metalloproteinase 1 (MTI-MMP). J Biol Chem, 1998. 273(2): p. 1216-22.

31. Hayakawa, T., et al., Cell growth-promoting activity of tissue inhibitor of metalloproteinases-2 (TIMP-2). J Cell Sci, 1994. 107(Pt 9): p. 2373-9.

32. Wang, X. S., et al., MAPKKK6, a novel mitogen-activated protein kinase kinase kinase, that associates with MAPKKK5. Biochem Biophys Res Commun, 1998. 253(1): p. 33-7.

33. Kratzschmar, J., L. Lunn, and C. P. Blobel, Metargidin, a membrane-anchored metalloprotease-disinitegrin protein with an RGD integrin binding sequence. J Biol Chem, 1996. 271(9): p. 4593-6.

34. Nath, D., et al., Interaction of metargidin (ADAM-15) with alphavbeta3 and alpha5beta1 integrins on different haemopoietic cells. J Cell Sci, 1999. 112(Pt4): p. 579-87.

35. Knecht, R., et al., Prognostic significance of polo-like kinase (PLK) expression in squamous cell carcinomas of the head and neck. Cancer Res, 1999. 59(12): p. 2794-7.

36. Smits, V. A., et al., Polo-like kinase-1 is a target of the DNA damage checkpoint. Nat Cell Biol, 2000. 2(9): p. 672-6.

37. Toyoshima-Morimoto, F., et al., Polo-like kinase 1 phosphorylates cyclin B1 and targets it to the nucleus during prophase. Nature, 2001. 410(6825): p. 215-20.

38. Schmucker, D. and S. L. Zipursky, Signaling downstream of eph receptors and ephrin ligands. Cell, 2001. 105(6): p. 701-4.

39. Miao, H., et al., Activation of EphA receptor tyrosine kinase inhibits the Ras/MAPK pathway. Nat Cell Biol, 2001. 3(5): p. 527-30.

40. Miao, H., et al., Activation of EphA2 kinase suppresses integrin function and causes focal-adhesion-kinase dephosphorylation. Nat Cell Biol, 2000. 2(2): p. 62-9.

41. Welsh, M., et al., Shb is a ubiquitously expressed Src homology 2 protein. Oncogene, 1994. 9(1): p. 19-27.

42. Oishi, K., et al., Identification and characterization of PKNbeta, a novel isoform of protein kinase PKN: expression and arachidonic acid dependency are different from those of PKNalpha. Biochem Biophys Res Commun, 1999. 261(3): p. 808-14.

43. Gu, M., I. Warshawsky, and P. W. Majerus, Cloning and expression of a cytosolic negakaryocyte protein-tyrosinie-phosphatase with sequence homology to retinaldehyde-binding protein and yeast SEC14p. Proc Natl Acad Sci U S A, 1992. 89(7): p. 2980-4.

44. Stebbins, C. E., W. G. Kaelin, Jr., and N. P. Pavletich, Structure of the VHL-ElonginC-ElonginB complex: implications for VHL tumor suppressor function. Science, 1999. 284(5413): p. 455-61.

45. Maxwell, P. H., et al., The tumor suppressor protein VHL targets hypoxia-inducible factors for oxygen-dependent proteolysis. Nature, 1999. 399(6733): p. 271-5.

46. Kibel, A., et al., Binding of the von Hippel-Lindau tumor suppressor protein to Elongin B and C. Science, 1995. 269(5229): p. 1444-6.

47. Sakano, S., et al., Molecular cloning of a novel non-receptor tyrosine kinase, HYL (hematopoietic consensus tyrosine-lacking kinase). Oncogene, 1994. 9(4): p. 1155-61.

48. Rane, M. J., et al., p38 Kinase-dependent MAPKAPK-2 activation functions as 3-phosphoinositide-dependent kinase-2for Akt in human neutrophils. J Biol Chem, 2001. 276(5): p. 3517-23.

49. Rouse, J., et al., A novel kinase cascade triggered by stress and heat shock that stimulates MAPKAP kinase-2 and phosphorylation of the small heat shock proteins. Cell, 1994. 78(6): p. 1027-37.

50. Gurney, M. E., et al., Neutroleukin: a lymphokine product of lectin-stimulated T cells. Science, 1986. 234(4776): p. 574-81.

51. Faik, P., et al., Mouse glucose-6-phosphate isomerase and ineuroleukin have identical 3′sequences. Nature, 1988. 332(6163): p. 455-7.

52. Chaput, M., et al., The neurotrophic factor neuroleukin is 90% homologous with phosphohexase isomerase. Nature, 1988. 332(6163): p. 454-5.

53. Gurney, M. E., et al., Molecular cloning and expression of neuroleukin, a neurotrophic factor for spinal and sensory neurons. Science, 1986. 234(4776): p. 566-74.

54. Wang, H., et al., Characterization of PCP-2, a novel receptor protein tyrosine phosphatase of the MAM domain family. Oncogene, 1996. 12(12): p. 2555-62.

55. Frigerio, J. M., J. C. Dagorn, and J. L. Iovanna, Cloning, sequencing and expression of the L5, L21, L27a, L28, S5, S9, S10 and S29 human ribosomal protein mRNAs. Biochem Biophys Acta, 1995. 1262(1): p. 64-8.

56. Yan, H., et al., A novel receptor tyrosine phosphatase-sigma that is highly expressed in the nervous system. J Biol Chem, 1993. 268(33): p. 24880-6.

57. Elchebly, M., et al., Neuroendocrine dysplasia in mice lacking protein tyrosine phosphatase sigma. Nat Genet, 1999. 21(3): p. 330-3.

58. Nagase, T., et al., Prediction of the coding sequences of unidentified human genes. IV The coding sequences of 40 new genes (KIAA0121-KIAA0160) deduced by analysis of cDNA clones from human cell line KG-1. DNA Res, 1995. 2(4): p. 167-74, 199-210.

59. Benovic, J. L., et al., Beta-adrenergic receptor kinase: primary structure delineates a multigene family. Science, 1989. 246(4927): p. 235-40.

60. Benovic, J. L., et al., Purification, and characterization of the beta-adrenergic receptor kinase. J Biol Chem, 1987. 262(19): p. 90²6-32.

61. Colamonici, O. R., et al., p135tyk2, an interferon-alpha-activated tyrosine kinase, is physically associated with an interferon-alpha receptor. J Biol Chem, 1994. 269(5): p. 3518-22.

62. Stahl, N., et al., Association and activation of Jak-Tyk kinases by CNTF-LIF-OSM-IL-6 beta receptor components. Science, 1994. 263(5143): p. 92-5.

63. Bernards, A., Predicted tyk2 protein contains two tandem protein kinase domains. Oncogene, 1991. 6(7): p. 1185-7.

64. Wang, B., et al., Molecular cloning and characterization of a novel human receptor protein tyrosine phosphatase gene, hPTP-J: down-regulation of gene expression by PMA and calcium ionophore in Jurkat T lymphoma cells. Biochem Biophys Res Commun, 1997. 231(1): p. 77-81.

65. Takeda, K., et al., Essential role of Stat6 in IL-4 signalling. Nature, 1996. 380(6575): p. 627-30.

66. Masuda, A., et al., Interleukin-15 Prevents Mouse Mast Cell Apoptosis through STAT6-mediated Bcl-xL Expression. J Biol Chem, 2001. 276(28): p. 26107-13.

67. Kato, Y., et al., BMK1/ERK5 regulates serum-induced early gene expression through transcription factor MEF2C. Embo J, 1997. c 16(23): p. 7054-66.

68. Kato, Y., et al., Bmk/Erk5 is required for cell proliferation induced by epidermal growth factor. Nature, 1998. 395(6703): p. 713-6.

69. Zhou, G., Z. Q. Bao, and J. E. Dixon, Components of a new human protein kinase signal transduction pathway. J Biol Chem, 1995. 270(21): p. 12665-9.

70. Siliciano, J. D., T. A. Morrow, and S. V. Desiderio, itk, a T-cell-specific tyrosine kinase gene inducible by interleukin 2. Proc Natl Acad Sci U S A, 1992. 89(23): p. 11194-8.

71. Tanaka, N., et al., A novel human tyrosine kinase gene inducible in T cells by interleukin 2. FEBS Lett, 1993. 324(1): p. 1-5.

72. Deak, M., et al., Mitogen- and stress-activated protein kinase-1 (MSK1) is directly activated by MAPK and SAPK2/p38, and may mediate activation of CREB. Embo J, 1998. 17(15): p. 4426-41.

73. Li, J., et al., Novel NEMO/IkappaB kinase and NF-kappa B target genes at the pre-B to immature B cell transition. J Biol Chem, 2001. 276(21): p. 18579-90.

74. Baytel, D., et al., The human Pin-2 proto-oncogene and its testicular expression. Biochem Biophys Acta, 1998. 1442(2-3): p. 274-85.

75. Wang, D., et al., Tumor necrosis factor alpha-induced phosphorylation of RelA/p65 on Ser529 is controlled by casein kinase II. J Biol Chem, 2000. 275(42): p. 32592-7.

76. Nada, S., et al., Constitutive activation of Src family kinases in mouse embryos that lack Csk Cell, 1993. 73(6): p. 1125-35.

77. Imamoto, A. and P. Soriano, Disruption of the csk gene, encoding a negative regulator of Src family tyrosine kinases, leads to neural tube defects and embryonic lethality in mice. Cell, 1993. 73(6): p. 1117-24.

78. Rothwaf, D. M., et al., IKK-gamma is an essential regulatory subunit of the IkappaB kinase complex. Nature, 1998. 395(6699): p. 297-300.

79. Millward, T. A., et al., Calciuim regulation of Ndr protein kinase mediated by S100 calcium-binding proteins. Embo J, 1998. 17(20): p. 5913-22.

80. Millward, T., P. Cron, and B. A. Hemmings, Molecular cloning and characterization of a conserved nuclear serine(threonine) protein kinase. Proc Natl Acad Sci U S A, 1995. 92(11): p. 5022-6.

81. Cheng, M., T. G. Boulton, and M. H. Cobb, ERK3 is a constitutively nuclear protein kinase. J Biol Chem, 1996. 271(15): p. 8951-8.

82. Boulton, T. G., et al., ERKs: a family of protein-serine/threonine kinases that are activated and tyrosine phosphorylated in response to insulin and NGF. Cell, 1991. 65(4): p. 663-75.

83. Hande, K. R., Clinical applications of anticancer drugs targeted to topoisomerase II. Biochem Biophys Acta, 1998.-1400(1-3): p. 173-84.

84. Wo, Y. Y., et al., Sequencing, cloning, and expression of human red cell-type acid phosphatase, a cytoplasmic phosphotyrosyl protein phosphatase. J Biol Chem, 1992. 267(15): p. 10856-65.

85. Haystead, T. A., et al., Effects of the tumor promoter okadaic acid on intracellular protein phosphorylation and metabolism. Nature, 1989. 337(6202): p. 78-81.

86. Kishikawa, K., et al., Phosphatidic acid is a potent and selective inhibitor of protein phosphatase I and an inhibitor of ceramide-mediated responses. J Biol Chem, 1999. 274(30): p. 21335-41.

87. Warbrick, E., The puzzle of PCNA's many partners. Bioessays, 2000. 22(11): p. 997-1006.

88. Tiganis, T., B. E. Kemp, and N. K. Tonks, The protein-tyrosine phosphatase TCPTP regulates epidermal growth factor receptor-mediated and phosphatidylinositol 3-kinase-dependent signaling. J Biol Chem, 1999. 274(39): p. 27768-75.

89. Tiganis, T., et al., Epidermal growth factor receptor and the adaptor protein p52Shc are specific substrates of T-cell protein tyrosine phosphatase. Mol Cell Biol, 1998.18(3): p. 1622-34.

90. Sanchez, Y., et al., Conservation of the Chk1 checkpoint pathway in mammals: linkage of DNA damage to Cdk regulation through Cdc25. Science, 1997. 277(5331): p. 1497-501.

91. Furnari, B., N. Rhind, and P. Russell, Cdc25 mitotic inducer targeted by chk1 DNA damage checkpoint kinase. Science, 1997. 277(5331): p. 1495-7. 92. Mailand, N., et al., Rapid destruction of human Cdc25A in response to DNA damage. Science, 2000. 288(5470): p. 1425-9. [symbol] res of fatty acid oxidation during reperfusion of ischemic hearts are associated with a decrease in malonyl-CoA [symbol] in 5′-AMP-activated protein kinase inhibition of acetyl-CoA carboxylase. J Biol Chem, 1995. 270(29): p. [symbol] tion of the human YVH1 protein-tyrosine phosphatase orthologue reveals a novel zinc binding domain [symbol] tion. J Biol Chem, 1999. 274(34): p. 23991-5. [symbol] (+)-like gene in human cells. Nature, 1991. 353(6339): p. 80-3. [symbol] and weel cooperate in the inhibitory tyrosine phosphorylationt of cdc2. Cell, 1991. 64(6): p. 1111-22. [symbol] Negative regulation of mitosis by weel+, a gene encoding a protein kinase homolog. Cell, 1987. 49(4): p. 559- [symbol] ular masking of nuclear import signal on NF-AT4 by casein kinase I and MEKKI. Cell, 1998. 93(5): p. 851-61. [symbol] n kinase I transduces Wnt signals. Nature, 1999. 401(6751): p. 345-50. [symbol] gg, Identification of 21 novel human protein kinases, including 3 members of a family related to the cell cycle [symbol] giillus nidulas. Cell Growth Differ, 1993. 4(10): p. 821-30. [symbol] I protein kinase phosphorylates human Cdc25C on serine 216 and promotes 14-3-3 protein binding. Cell [symbol] : p. 197-208. [symbol] Identification and characterization of a human protein kinase related to budding yeast Cdc7p. Proc Natl Acad [symbol] p. 14320-5. [symbol] vel SR-protein-specific kinase, SRPK2, disassembles nuclear speckles. Biochem Biophys Res Commun, 1998. [symbol] 2: a differentially expressed SR protein-specific kinase involved in mediating the interaction and localization of [symbol] rs in mammalian cells. J Cell Biol, 1998. 140(4): p. 737-50. [symbol] n of apoptosis by ASKI, a mammalian MAPKKK that activates SAPK/JNK and p38 signaling pathwvays. [symbol] ): p. 90-4. [symbol] vel kinase cascade mediated by mitogen-activated protein kinase kinase 6 and MKK3. J Biol Chem, 1996. [symbol] ion of the c-Jun N-terninal kinase pathway by a novel protein kinase related to human germinal center kinase. [symbol] 1997. 94(18): p. 9687-92. [symbol] J M. W. Kilimann, Structure of the human gene encoding the phosphorylase kinase beta subunit (PHKB). Eur J [symbol] 374-80.

109. Hobert, O., B. Jallal, and A. Ullrich, Interaction of Vav with ENX-1, a putative transcriptionial regulator of homeobox gene expression. Mol Cell Biol, 1996.16(6): p. 3066-73.

110. Hobert, O., et al., Isolation and developmental expression analysis of Enx-1, a novel mouse Polycomb group gene. Mech Dev, 1996. 55(2):,p. 171-84.

11. Alexandru, G., et al., Sister chromoatid separation and chromosonme re-duplication are regulated by different mechanisms in response to spindle (damage. Embo J, 1999. 18(10): p. 2707-21.

112. Cahill, D. P., et al., Mutations of mitotic checkpoint genes in human cancers. Nature, 1998. 392(6673): p. 300-3.

113. Schultz, S. J., et al., Cell cycle-dependent expression of Nek2, a novel human protein kinase related to the NIMA mitotic regulator of Aspergillus nidulans. Cell Growth Differ, 1994. 5(6): p. 625-35.

114. Fry, A. M., P. Meraldi, and E. A. Nigg, A centr-osomlalfunition for the human Nek2 protein kinase, a member of the NIMA family of cell cycle regulators. Embo J, 1998. 17(2): p. 470-81.

115. Zhao, Y., et al., Cloning and chromosomal location of a novel member of the myotoitic dystrophy family of protein kinases. J Biol Chem, 1997. 272(15): p. 10013-20.

116. Tung, R. M. and J. Blenis, A novel human SPSI/STE20 homologue, KHS, activates Jun N-terminal kinase. Oncogene, 1997. 14(6): p. 653-9.

117. Yuan, Y., D. M. Li, and H. Sun, PIR1, a novel phosphatase that exhibits high affinity to RNA. ribonucleoprotein complexes. J Biol Chem, 1998. 273(32): p. 20347-53.

118. Deshpande, T., et al., Human PIR1 of the protein-tyrosine phosphatase superfamily has RNA 5′-triphosphatase and diphosphatase activities. J Biol Chem, 1999. 274(23): p. 16590-4.

119. Bastians, H. and H. Ponstingl, The novel human protein serine/threonine phosphatase 6 is a functional homologue of budding yeast Sit4p and fission yeast ppel, wjocj are involved in cell cycle regulation. J Cell Sci, 1996. 109(Pt 12): p. 2865-74.

120. Becker, W., et al., Sequence characteristics, subcellular localization, and substrate specificity of DYRK-related kinases, a novel family of dual specificity protein kinases. J Biol Chem, 1998. 273(40): p. 25893-902.

121. Guimera, J., et al., A human homologue of Drosophila minibrain (MNB) is expressed in the neuronal regions affected in Down syndrome and maps to the critical region. Hum Mol Genet, 1996. 5(9): p. 1305-10.

122. Lopez-Borges, S. and P. A. Lazo, The human vaccinia-related kinase I (VRK1) phosphorylates threonine-18 within the mdm-2 binding site of the p53 tumour suppressor protein. Oncogene, 2000. 19(32): p. 3656-64.

123. Nezu, J., et al., Identification of two novel human putative serine/thireonine kinases, VRK1 and VRK2, with structural similarity to vaccinia virus BIR kinase. Genomics, 1997. 45(2): p. 327-31.

124. Lapidot-Lifson, Y., et al., Cloning and antisense oligodeoxynucleotide inhibition of a human homolog of cdc2 required in hematopoiesis. Proc Natl Acad Sci U S A, 1992. 89(2): p. 579-83.

125. Mills, G. B., et al., Expression of TTK, a novel human protein kinase, is associated with cell proliferation. J Biol Chem, 1992. 267(22): p. 16000-6.

126. Schmandt, R., et al., IL-2-induced expression of TTK, a serine, threonine, tyrosine kinase, correlates with cell cycle progression. J Immuniol, 1994. 152(1): p. 96-105.

127. Ruvolo, P. P., et al., Ceramide induces Bcl2 dephosphorylation via a mechanism involving mitochondrial PP2A. J Biol Chem, 1999. 274(29): p. 20296-300.

128. Wadzinski, B. E., et al., Nuclear protein phosphatase 2A dephosphorylates protein kinase A-phosphorylated CREB and regulates CREB transcriptional stimulation. Mol Cell Biol, 1993. 13(5): p. 2822-34.

129. Lee, M. G. and P. Nurse, Complementation used to clone a human homologue of the fission yeast cell cycle control gene cdc2. Nature, 1987. 327(6117): p. 31-5.

130. Riabowol, K., et al., The cdc2 kinase is a nuclear protein that is essential for mitosis in mammalian cells. Cell, 1989. 57(3): p. 393-401.

131. Zeng, Q., et al., Pretylation-dependent association of protein-tyrosine phosphatases PRL-1, -2, and -3 with the plasma membrane and the early endosome. J Biol Chem, 2000. 275(28): p. 21444-52.

132. Diamond, R. H., et al., PRL-1, a unique nuclear protein tyrosine phosphatase, affects cell growth. Mol Cell Biol, 1994. 14(6): p. 3752-62.

133. Peng, Y., et al., The gene encoding human nuclear protein tyrosine phosphatase, PRL-1. Cloning, chromosomal localization, and identification of an intron enhancer, J Biol Chem, 1998. 273(27): p. 17286-95.

134. Edwards, M. C., C. Wong, and S. J. Elledge, Human cyclin K, a novel RNA polymerase II-associated cyclini possessing both carboxy-terminal domain kinase and Cdk-activating kinase activity. Mol Cell Biol, 1998. 18(7): p. 4291-300.

135. van den Berg, I. E., et al., X-linked liver phosphorylase kinase deficiency is associated with mutations in the human liver phosphorylase kinase alpha subunit. Am J Hum Genet, 1995. 56(2): p. 381-7.

136. Grove, J. R., et al., Cloning and expression of two human p⁷⁰ S6 kinase polypeptides differing only at their amino termini. Mol Cell Biol, 1991. 11(11): p. 5541-50.

137. Banerjee, P., et al., Molecular structure of a major insulin/mitogen-activated 70-kDa S6 protein kinase. Proc Natl Acad Sci U S A, 1990. 87(2l)- p. 8550-4.

138. Weng, Q. P., et al., Phosphatidylinositol 3-kinase signals activation of p70 S6 kinase in situ through site-specific p70 phosphorylation. Proc Natl Acad Sci U S A, 1995. 92(12): p. 5744-8.

139. Kornbluth, S., M. Sudol, and H. Hanafusa, Association of the polyomavirus middle-T antigen with c-yes protein. Nature, 1987. 325(7000): p. 171-3.

140. Kitamura, N., et al., Avian sarcoma virus Y73 genome sequence and structural similarity of its transforming gene product to that of Rous sarcoma virus. Nature, 1982. 297(5863): p. 205-8.

141. Tobimatsu, T. and H. Fujisawa, Tissue-specific expression of four types of rat calmodulin-dependent protein kinase II mRNAs. J Biol Chem, 1989. 264(30): p. 17907-12.

142. Hovens, C. M., et al., RYK, a receptor tyrosine kinase-related molecule with unusual kinase domain motifs. Proc Natl Acad Sci U S A, 1992. 89(24): p. 11818-22.

143. Halford, M. M., et al., Ryk-deficient mice exhibit craniofacial defects associated with perturbed Eph receptor crosstalk. Nat Genet, 2000. 25(4): p. 414-8.

144. Barker, H. M., et al., Sequence of human protein serine/threonine phosphatase 1 gamma and localization of the gene (PPPICC) encoding it to chromosone bands 12q24.1-q24.2. Biochem Biophys Acta, 1993. 1178(2): p. 228-33.

TABLE 1
Genes that have not been linked to an
antiapoptotic function before and are
Induced in the apoptosis resistant clones

| | | Number of clones | |
|---|---|---|---|
| | | in which | |
| | | expression | |
| | | exceeds cut off | |
| | Accession | for increased | % |
| Gene | number | expression | over cutoff |
| Tyrosine Kinases | | | |
| Hck | M16591 | 4 | 46.5 |
| TrkC | U05012 | 6 | 28.0 |
| Hyl | X77278 | 11 | 24.5 |
| Rse | U05682 | 10 | 22.5 |
| RON | X70040 | 14 | 21.0 |
| KIAA0641 | AB014541 | 5 | 19.5 |
| EphA2 | M59371 | 12 | 19.5 |
| Csk | X59932 | 8 | 18.0 |
| EphB3 | X75208 | 6 | 16.0 |
| EphB4 | U07695 | 5 | 12.5 |
| Pyk-2 | U33284 | 4 | 11.0 |
| Unknown Phosphatases | | | |
| PB-26 | AB040904 | 5 | 30.5 |
| PB-28 | AB040904 | 4 | 18.0 |
| Unknown Kinases | | | |
| UK19 | AA292586 | 5 | 25.0 |
| UK10 | R52045 | 4 | 24.5 |
| UK11 | H39075 | 8 | 9.5 |
| Serine/Threonine | | | |
| Kinases | | | |
| GRK6 | L16862 | 16 | 69.0 |
| Dyrk4 | Y09305 | 5 | 55.0 |
| IRAK-2 | AF026273 | 13 | 54.5 |
| LIMK-1 | D26309 | 5 | 45.5 |
| MLK3 | U07747 | 9 | 44.0 |
| AMPK-beta | AJ224538 | 4 | 40.0 |
| MAPKKK6 | AF100318 | 14 | 39.0 |
| MAST205 | 6678957 | 14 | 37.5 |
| DAPK | X76104 | 11 | 37.0 |
| MAPKK3 | 4506098 | 11 | 36.0 |
| PLK-1 | L19559 | 12 | 34.5 |
| PKN-H4 | D26181 | 13 | 34.5 |
| Bcr | X02596 | 10 | 32.0 |
| MSK2 | AF074715 | 8 | 30.0 |
| Rac-alpha | M63167 | 16 | 28.0 |
| MST-3 | AF024636 | 7 | 28.0 |
| PSK-H1 | M14504 | 5 | 26.5 |
| PCTAIRE1 | X66363 | 6 | 23.0 |
| lok | AB015718 | 4 | 22.0 |
| HsGAK | D88435 | 6 | 21.0 |
| MAPKAPK3 | U09578 | 7 | 20.5 |
| JNKK2alpha | AF022805 | 8 | 19.5 |
| FAST | X86779 | 5 | 19.0 |
| MKK7 | AF013588 | 8 | 17.5 |
| MAPKK5 | U25265 | 6 | 16.5 |
| PAK1-relatedkinase | AF005046 | 11 | 16.0 |
| ARK2 | AF008552 | 4 | 16.0 |
| MSSK1 | U82808 | 4 | 15.0 |
| PHK-gammaT | M31606 | 6 | 14.5 |
| CDC42- | AF128625 | 3 | 13.5 |
| bindingproteinkinasebeta | | | |
| KIAA0151 | D63485 | 9 | 11.0 |
| KIAA0537 | AB011109 | 6 | 11.5 |
| STE20-like | X99325 | 4 | 6.0 |
| Ste-20likeproteinkinase3 | AF083420 | 3 | 5.5 |
| Adapter Proteins | | | |
| Grb-2 | M96995 | 7 | 18.0 |
| SHC | Y09847 | 6 | 17.5 |
| SHB | X75342 | 10 | 15.0 |
| Phosphatases | | | |
| PYST1 | X93920 | 5 | 85.5 |
| B23 | U15932 | 5 | 33.0 |
| PCP-2 | X97198 | 10 | 31.5 |
| PTP-J | U73727 | 9 | 30.0 |
| PTP-Meg2 | M83738 | 9 | 29.5 |
| PP5 | X89416 | 7 | 18.5 |
| CDC25B | M81934 | 10 | 17.0 |
| PTP-SL | Z30313 | 5 | 17.0 |
| PP2B-R | M30773 | 6 | 17.0 |
| PP1-Calpha | M63960 | 6 | 16.0 |
| PP2A-Rb55 | M64930 | 5 | 15.5 |
| PTPzeta | X54135 | 4 | 13.0 |
| Shp-1 | X62055 | 4 | 7.0 |
| PP2A-Ra65 | J02902 | 6 | 7.0 |
| PTPmu | X58288 | 3 | 6.5 |
| Metalloproteases | | | |
| MMP-15 | Z48482 | 19 | 71.0 |
| ADAM12 | X05232 | 8 | 59.0 |
| MMP-3 | J03209 | 14 | 34.5 |
| ADAM15 | NM003815 | 12 | 28.0 |
| ADAM8 | XM005675 | 13 | 27.0 |
| G-proteins | | | |
| alphai2 | | 16 | 44.0 |
| GPIR-3 | | 4 | 8.0 |
| Other | | | |
| p91/ISGF-3 | M97935 | 7 | 104.0 |
| 90k | 50318862 | 15 | 57.5 |
| MHC-1 | M11886 | 17 | 53.0 |
| EF-2 | X51466 | 16 | 44.0 |
| alpha-tubulin | NM_006082 | 9 | 43.5 |
| KIF-1c | NM006612 | 6 | 28.0 |
| Furin | X17094 | 8 | 27.0 |
| rS9 | 4506744 | 13 | 23.0 |
| GPDH | M33197 | 9 | 23.0 |
| beta-Aktin | X00351 | 11 | 21.5 |
| Vimentin | X56134 | 15 | 19.0 |
| neurolektin | K03515 | 14 | 17.5 |
| Thymosinbeta | S54005 | 7 | 11.5 |
| Histon3.3 | M11354 | 6 | 7.0 |
| PHB-4-PC | L14273 | 5 | 3.5 |

TABLE 2
Genes with known antiapoptotic function that are
Induced in the apoptosis resistant clones

| | | Number of clones in which | |
|---|---|---|---|
| | Accession | expression exceeds cut off | % over |
| Gene | number | for increased expression | cut off |
| Tyrosine Kinases | | | |
| PDGFRalpha | M22734 | 4 | 39.0 |
| HER2 | M11730 | 4 | 38.5 |
| EGFR | X00588 | 6 | 21.0 |
| FGFR-3 | M58051 | 5 | 20.5 |
| HER4 | L07868 | 5 | 32.5 |
| Jak-2 | AF058925 | 6 | 34.0 |
| Tyk-2 | X54637 | 8 | 18.5 |
| Serine/Threonine | | | |
| Kinases | | | |
| RSK | L07597 | 11 | 43.5 |
| MAPKK2 | L11285 | 11 | 33.5 |
| PIM-2h | U77735 | 8 | 29.5 |
| IKK1 | AF012890 | 4 | 28.0 |
| CKII-beta | M30448 | 7 | 28.0 |
| ALK-4 | Z22536 | 6 | 27.0 |
| ERK1 | X60188 | 11 | 25.5 |
| IKKgamma | AF074382 | 12 | 23.5 |
| AKT2 | M95936 | 10 | 17.0 |
| CKII-alpha | J02853 | 8 | 17.5 |
| CaM-KIIgamma | L07044 | 7 | 16.0 |
| MAPKAPK2 | NM004759 | 6 | 15.0 |
| ILK | U40282 | 7 | 14.0 |
| CKI-delta | U29171 | 6 | 11.5 |
| SGK | Y10032 | 4 | 11.5 |
| CKII-beta | M30448 | 5 | 9.0 |
| A-Raf-1 | X04790 | 5 | 9.0 |
| ALK-1 | L17075 | 5 | 6.5 |
| Phosphatases | | | |
| PPX | X70218 | 16 | 26.0 |
| Ligands | | | |
| TGFalpha | XM002732 | 11 | 63.0 |
| IL1-beta | NM000576 | 8 | 54.5 |
| IL1-alpha | X02531 | 4 | 34.5 |
| VEGF | NM003376 | 9 | 27.0 |
| Other | | | |
| Bcl-x | Z23115 | 14 | 34.5 |
| IL-4Stat | U16031 | 9 | 19.5 |
| TIMP-1 | X03124 | 17 | 108.5 |
| myc | X00364 | 8 | 15.5 |
| TIMP-2 | S48568 | 13 | 36.5 |

TABLE 3
Clusters in the apoptosis resistant clones
Bold: also found in one common cluster in squamous cell carcinoma
Cluster 1

| | |
|---|---|
| Correlation factor 0.71 | |
| Bcr | Rac-alpha |
| JNKK2alpha | IKKgamma |
| MKK7 | PPX |
| PTP-SL | ADAM15 |
| KIF-1c | ADAM8 |
| SHB | ERK1 |
| IRAK | Rse |
| PLK-1 | alphai2S48C |
| alpha-tubulin | MAPKKK6 |
| RSK | 90k |
| GRK6 | pBTUB |
| GPIR-3 | |
| MAPKAPK3 | |
| PKN-H4 | |
| MAPKK2 | |
| PKA-RIbeta | |
| p130CAS | |
| LIMK-1 | |
| MAST205 | |
| PIM-2h | |
| Csk | |
| MAPKK3 | |
| PCTAIRE1 | |
| MST-3 | |

Cluster 2

Correlation factor 0.73
Bcl-x
AKT2
EF-2
PITALRE
TIMP-2
FGFR-2
CDC25B
CKI-delta
EphA2
PP2C
Rac-alpha
RON
EphB4

Cluster 3

Correlation factor 0.74
PDGFRalpha
PB-28
PHK-gammaT
Tyk-2
A-Raf-1
GPDH
HK-18B
Erk6
Cyto18
HK-18B
cytokeratin8
CK-8
CKII-beta
MLK3
MMP-11

Cluster 4

Correlation factor 0.67
PP2A-Ra65
HER2
CAMKK

Cluster 5

Correlation factor 0.71
PKC-epsilon
MAPKK5
PTP-Meg2
PP2B-Cbeta
MKP-5
Jak-2
Shp-2
IKK2
PHK-alphaL
JNK1

Cluster 6

Correlation factor 0.61
Pyk-2
Shp-1
PP2B-Cgamma
lok
CaM-KIIgamma
DRP-1
CKI-gamma2
PSK-H1
GPIR-1
Chk2
Axl
IL-4Stat
PCP-2
DAPK
PKA-Calpha2
PP5
Abl
Raf
PTPzeta
IGF1-R
pHE-A1
PTP-1B
MSTH1
PKA-Calpha
PLCgamma
HsGAK
VHR
TESK1
PRK

Cluster 7

Correlation factor 0.83
MMP-15
TIMP-1

TABLE 4
Clusters in squamous cell carcinoma cell lines
SCaBER
UMSSC-17B
UMSSC-17A
UMSSC-22A
UMSSC-22B
UMSSC-10A
HlaC78
HlaC79
FaDu
Bold: also found in one common cluster in apoptosis resistant clones

Cluster 1

Correlation factor 0.7
PRL-3_2_23
Dyrk2_2_16
MAPKK2_1_5
SHC_1_3
ERK3_1_4
RSK

Cluster 2

Correlation factor 0.8
GSK-3alpha
PTP-SL
MAPKAPK3
GSK-3beta
hPAK1
PKC-delta
PB-32
alphaq
KIF-1c
SHB
KIAA0687Nck-Interactingkinase

Cluster 3

Correlation factor 0.67
PB-32
PB-38
hPAK2
TIMP-2
PB-5
CDK6
Bcl-x
H11
Axl
MMP-14
hSLK
MSTH1
alphaq
ADAM17
PIR1
ALK-2

Cluster 4

Correlation factor 0.71
Tyk-2
TIMP-1
GPDH
HPRT
Dyrk4
MMP-15
Jak-1
Myt1
GPIR-3
PCNA
Chk2
PK38
alpha-tubulin
CDK4
PKN-H4
hPTK
GPIR-2
Bmx
MMP-11
PKU-alpha

TABLE 5

| | | | | Number of | |
|---|---|---|---|---|---|
| | | | | clones with | % over cut off for |
| | Genbank | | | increased | increased |
| Gene | Nr. | Description | Reference | expression | expression |
| MP_MMP-15 | Z48482 | transmembrane metalloprolease, probably processes | [1, 2] | 20 | 82 |
| | | MMP-2 | | | |
| STP_PPX | X70218 | nuclear, localized to centrosomes, activates NFkB by | [3] | 17 | 18 |
| | | dephosphorylation | | | |
| OT_MHC-1 | M11886 | presents antigens on the cell surface | [4] | 16 | 44 |
| OT_EF-2 | XM031904 | translation-elongation-factor-2 | [5] | 15 | 30 |
| OT_Vimentin | X56134 | intermediate filament | [6] | 15 | 14 |
| GP_alphai2 | NM002070 | alpha subunit of heterotrimeric G-proteins, can inhibit | [7-10] | 14 | 42 |
| | | adenylate cyclase and activate MAP-kinase | | | |
| STK_PKNalpha | D26181 | related to PKC, activated by Rho, fatty acids and | [11-15] | 14 | 34 |
| | | caspase cleavage | | | |
| STK_Bcr | X02596 | possesses serine/threonine kinase activity and GAP | [16, 17] | 13 | 32 |
| | | activity for p21rac | | | |
| STYP_CDC25B | M81934 | dual specific, induces cell cycle progression form G2 | [18-21] | 13 | 30 |
| | | to M by dephosphorylation of cdc-2 | | | |
| STP_PP5_1_3 | X89416 | nuclear, binds to the glucocorticoid receptor and | [22-24] | 13 | 22 |
| | | inhibits growth inhibition by this receptor | | | |
| STK_MAST205 | 6678957 | binds to microtubuli and β2-syntrophin | [25, 26] | 13 | 20 |
| MP_ADAM8 | NM001109 | induced by TNF-α | [27, 28] | 13 | 18 |
| MP_TIMP-2 | S48568 | contributes to the activation of pro-Gelatinase A in | [29-31] | 12 | 37 |
| | | complex with MMP-14, may act mitogenically | | | |
| STK_MAPKKK6 | U39657 | binds MAPKKK5/ASK1 | [32] | 12 | 33 |
| MP_ADAM15 | NM003815 | binds to Integrins | [33, 34] | 12 | 32 |
| STK_PLK-1 | L19559 | prognostic marker for squamous cell carcinoma, | [35-37] | 12 | 21 |
| | | essential for Pro-phase of mitosis, acitvated after DNA | | | |
| | | damage | | | |
| YK_EphA2 | M59371 | plays a role in repulsion of nerve cells during | [38-40] | 12 | 16 |
| | | embryogenisis, inhibits MAPK-activation by PDGF and | | | |
| | | EGF | | | |
| AD_SHB | NM003028 | SH2 domain containing adaptor protein | [41] | 12 | 15 |
| STK_PKNbeta | AB019692 | homologues to PKNalpha, not expressed in adult | [42] | 11 | 49 |
| | | healthy tissues but in cancer | | | |
| YP_PTP-Meg2 | M83738 | cytosolic tyrosine phosphatase | [43] | 11 | 42 |
| OT_VHL | NM_000551 | tumor suppressor that forms a complex with ubiquitin | [44-46] | 11 | 40 |
| | | ligase | | | |
| YK_Hyl | X77278 | cytoplasmic tyrosine kinase with homology to CSK | [47] | 11 | 18 |
| STK_MAPKAPK2 | NM032960 | activated by p38 MAP kinase, can act as PDK2 for Akt | [48, 49] | 11 | 10 |
| OT_neuroleukin | K03515 | neurotrophic ligand, entire mRNA also codes for | [50-53] | 11 | 10 |
| | | Phosphohexose-Isomerase | | | |
| YP_PCP-2 | X97198 | receptor tyrosine phosphatase with MAM domain | [54] | 10 | 30 |
| OT_rS9 | 4506744 | ribosomal protein 9 | [55] | 10 | 16 |
| YP_PTPsigma | U35234 | receptor tyrosine phosphatase of the LAR family, | [56, 57] | 9 | 109 |
| | | involved in brain embryogenisis | | | |
| STK_KIAA0135 | D50925 | putative Serin/Threonin-Kinase | [58] | 9 | 33 |
| STK_beta-ARK-1 | X61157 | phosphorylates and desensitizes β-adrenergic | [59, 60] | 9 | 25 |
| | | receptor | | | |
| YK_Tyk-2 | X54637 | cytoplasmic tyrosine kinase, homologues to JAK- | [61-63] | 9 | 20 |
| | | kinases | | | |
| YP_PTP-J | U73727 | receptor tyrosine phosphatase with MAM domain | [64] | 9 | 19 |
| OT_IL-4Stat | U16031 | transduces IL-4 signals | [65, 66] | 9 | 18 |
| STK_MAPKK5 | U25265 | activates Erk5/Bmk | [67-69] | 9 | 7 |
| YK_ITK | D13720 | cytoplasmic tyrosine kinase, specific for T-cells | [70, 71] | 8 | 46 |
| STK_MSK2 | AF074393 | activated by p38 and Erk1/2 Map-Kinases | [72] | 8 | 30 |
| STK_PIM-2h | U77735 | upregulated by NFkB | [73, 74] | 8 | 18 |
| STK_CKII-alpha | J02853 | activated by phosphorylation of ret/p65 | [75] | 8 | 17 |
| YK_Csk | X59932 | phosphorylates and inhibits src-kinases | [76, 77] | 8 | 15 |
| STK_IKKgamma | AF074382 | part of the IkappaB-Kinase complex that activates | [78] | 8 | 13 |
| | | NFkB | | | |
| TABLE 5 | | | | | |
| | | | | Number of | |
| | | | | Clones with | % under cut off for |
| | Genbank | | | reduced | reduced |
| Gene | Nr. | Description | Reference | expression | expression |
| STK_Ndr | Z35102 | nuclear phosphatase, activated by Calcium | [79, 80] | 20 | 59 |
| STK_ERK3 | X80692 | constitutively nuclear MAP-Kinase | [81, 82] | 19 | 159 |
| OT_Topoisomerase2 | NM001068 | topoisomerase-2 inhibitors are used as | [83] | 19 | 143 |
| | | chemotherapeutica against cancer | | | |
| YP_AZP- | M83653 | cytoplasmic phosphotyrosyl protein phosphatase | [84] | 19 | 92 |
| ISredacidphosphatase | | | | | |
| STP_PP1-Cbeta | X80910 | catalytic subunit of PP1, activated by ceramid | [85, 86] | 19 | 45 |
| OT_PCNA | 4505640 | Proliferating Cellular Nuclear Antigen | [87] | 19 | 27 |
| YP_TC-PTP | M25393 | localized to ER and nucleus, inhibits PI3K signals | [88, 89] | 19 | 155 |
| | | after EGF stimulation | | | |
| STK_CHK1 | AF016582 | after DNA damage necessary for cell cycle halt at | [90-92] | 18 | 104 |
| | | G2/M, phosphorylates wee1 and cdc25 | | | |
| STK_AMP- | AF100763 | phosphorylates and deactivates Acetyl-CoA | [93] | 18 | 81 |
| activatedproteinkinase | | Carboxylase | | | |
| alpha1subunit | | | | | |
| PP_YVH1 | AF119226 | dual-specific phosphatase | [94] | 18 | 72 |
| STK_WEE1 | X62048 | inhibits G2/M progression, phosphorylates and inhibits | [95-97] | 18 | 73 |
| | | cdcd 2 | | | |
| STK_CKI-alpha | X80693 | part of the Wnt pathway, phosphorylates and inhibits | [98, 99] | 18 | 49 |
| | | nuclear transport of NF-AT4 | | | |
| STK_NEK3 | Z29067 | Homologous to NIMA kinase of Aspergillus Nidulans, | [100] | 17 | 234 |
| | | which is responsible for G2/M progression | | | |
| STK_MAD-3likePK | AF068760 | | | 17 | 104 |
| STK_TAK1 | U64205 | cdc25 associated kinase, phosphorylates Cdc25c | [101] | 17 | 53 |
| UP_PB-32 | W30715 | unknown phosphatase | | 16 | 178 |
| STK_HsCdc7 | AF015592 | important for G1/S progression | [102] | 16 | 138 |
| STK_SRPK-2 | U88666 | phosphorylates SR-Splice-factors | [103, 104] | 16 | 70 |
| STK_MAPKK6 | U39657 | activates p38 MAP-kinase, activated by Ask-1 a | [105, 106] | 16 | 54 |
| | | MAPKKK that induces apoptosis | | | |
| STK_GCK | U07349 | homologous to S. cerevisiae Ste20, activates JNK | [107] | 16 | 324 |
| STK_KIAA0619 | AB014519 | unknown kinase | | 16 | 239 |
| STK_PHK-beta | X84908 | phosphorylates Glycogen-Phosphorylase | [108] | 15 | 246 |
| OT_33a_Enx-1 | AF070418 | regulates expression of Homeobox-genes | [109, 110] | 15 | 187 |
| STK_Bub1 | AF046078 | controls segregation of chromatids, mutation in cancer | [111, 112] | 15 | 121 |
| | | causes increased mutation rate | | | |
| STK_NEK2 | U11050 | associates with centrosomes | [113, 114] | 15 | 37 |
| STK_PK428 | U59305 | related to family of myotonic dystrophy kinases | [115] | 15 | 237 |
| STK_KHS | U77129 | homologous to S. cerevisiae Ste20, activates JNK | [116] | 15 | 121 |
| PP_PIR1 | AF023917 | dual specific, nuclear, dephosphorylates RNA, | [117, 118] | 15 | 51 |
| | | associated with speckles | | | |
| STP_PP6 | X92972 | homologous to S. cerevisiae Sit4p and S. pombe | [119] | 15 | 30 |
| | | ppe1, which regulate the cell cycle | | | |
| STK_MNB | U52373 | dual specific, homologous to DYRK kinase, located in | [120, 121] | 14 | 90 |
| | | region of chromosome 21 that is amplified in Down- | | | |
| | | Syndrom | | | |
| STK_VRK1 | AB000449 | homologous to Vaccinia Virus Kinase, nuclear | [122, 123] | 14 | 286 |
| STK_CHED | M80629 | Homolog of cdc-2 | [124] | 14 | 75 |
| STK_TTK | M86699 | dual specific, expression correlates with cell cycle | [125, 126] | 13 | 465 |
| UK_PB-11 | AF061944 | unknown kinase | | 13 | 168 |
| STP_PP2A-Cbeta | X12656 | nuclear, dephosphorylates Bcl-2 | [127, 128] | 13 | 33 |
| UK_UK20 | NM_016507 | unknown kinase | | 13 | 282 |
| STK_GLK | AF000145 | homologous to S. cerevisiae Ste20, activates JNK | [107] | 13 | 140 |
| STK_26b_CDC2_1_4 | X05360 | essential for G2/M progression | [96, 129, 130] | 12 | 168 |
| YP_Prl-1 | U48297 | may influence cell growth, nuclear but also associated | [131-133] | 12 | 74 |
| | | with plasma membranes and endosomes | | | |
| STK_cyclinK | AF060515 | can regulate cdk-activity and transcription by RNA- | [134] | 12 | 31 |
| | | polymeerase II | | | |
| STK_PHK-alphaL | X80497 | subunit of phosphorylase kinase | [135] | 12 | 24 |
| STK_p70S6K | M60724 | activated via PI3Kinase | [136-138] | 11 | 824 |
| YK_Yes | 4885660 | belongs to family of src-kinases | [139, 140] | 11 | 661 |
| STK_CaM-Klldelta | U73504 | highly expressed in brain | [141] | 11 | 216 |
| YK_Ryk | X69970 | receptor tyrosine kinase, doesn't belong to any known | [142, 143] | 11 | 166 |
| | | family of receptor tyrosine kinases, probably involved | | | |
| | | in Eph-signalling | | | |
| YP_PRL-3 | AF041434 | homologous to PRL-1 | [131] | 11 | 74 |
| STP_PP1-Cgamma | X74008 | catalytic subunit of PP1 | [144] | 11 | 59 |

1. A method for identifying nucleic acid molecules functionally associated with a desired phenotype comprising the steps:
(a) providing a population of parental cells wherein said cell population substantially lacks the desired phenotype and wherein parental cells are selected, which are continuously in a process of genome rearrangement and mutagenesis,
(b) subjecting said cell population to a procedure resulting in a rearrangement and/or mutation of the cell genome,
(c) subjecting said cell population from (b) to a selection procedure for the desired phenotype,
(d) identifying and optionally characterizing cells exhibiting said desired phenotype,
(e) obtaining mRNA from cells exhibiting said desired phenotype,
(f) hybridizing said mRNA or cDNA derived therefrom to a nucleic acid array and determining gene expression in cells exhibiting said desired phenotype and
(g) comparing gene expression in cells exhibiting said desired phenotype with gene expression in cells substantially lacking the desired phenotype.

2. The method of claim 1 wherein the desired phenotype is selected from cancer cell properties.

3. The method of claim 2 wherein the cancer cell properties are selected from invasiveness, metastasis, loss of contact inhibition, loss of extracellular matrix requirement, growth factor independence, angiogenesis induction, immuno defense evasion and/or anti-apoptosis.

4. The method. of claim 2 wherein the desired phenotype is anti-apoptosis.

5. The method of claim 1 wherein the desired phenotype is selected from production of secreted protein, susceptibility or resistance to pathogens, senescene and regulation of cell functions.

6. The method of claim 1 wherein the parental cell is an immortalized or transformed cell.

7. The method of any one of claims 1-6 wherein step (d) comprises a cell sorting procedure.

8. The method of claim 7 wherein said cell sorting procedure is a Fluorescence Activated Cell Sorting Procedure (FACS).

9. The method of any one of claims 1-8 comprising obtaining mRNA in step (e) and hybridizing said mRNA or a nucleic acid made therefrom with a nucleic acid array.

10. The method of claim 9 wherein the nucleic acid made from mRNA is selected from the group consisting of cDNA and cRNA.

11. The method of any one of claims 9-10 wherein said nucleic acid array comprises a solid carrier having immobilized thereto a plurality of different nucleic acid molecules.

12. The method of any one of claims 9-11 wherein said nucleic acid array is selected from arrays of genomic DNA arrays, cDNA arrays and oligonucleotide arrays.

13. The method of any one of claims 9-12 wherein said nucleic acid array comprises nucleic acids encoding functional cellular polypeptides or portions thereof selected from kinases, phosphatases, enzymes and receptors.

14. The method of any one of claims 1-13 comprising obtaining protein in step (e) and analyzing the protein content in cells exhibiting the desired phenotype.

15. The use of claim 14 wherein said analyzing comprises 2D gel electrophoresis, mass spectrometry and/pr binding to protein arrays.

16. The method of claims 14 or 15 wherein before analyzing a pretreatment step in order to reduce the complexity of the protein mixture is carried out.

17. The method of any one of claims 1-16 further comprising the identification of a plurality of genes (gene cluster) which is associated with the desired phenotype.

18. The method of any one of claims 1-17 further comprising a validation step wherein the association of a defined gene or gene cluster with the desired phenotype is determined.

19. The method of claim 18 wherein the validation step comprises generating of dominant-negative mutants.

20. The method of any one of claims 1-19 further comprising a screening procedure wherein the activity of a test substance for a defined gene or gene cluster associated with the desired phenotype is determined.

21. Use of the method of any one of claims 1-20 for generating expression profiles of genes or gene clusters associated with a desired phenotype.

22. The use of claim 21 wherein the expression profile is compared with the expression profile in a biological sample.

23. The use of claim 22 wherein the sample is derived from a human patient.

24. Use of the nucleic acids as shown in Table 1 and Table 5 or fragments thereof or peptides or polypeptides encoded by said nucleic acids or fragments or combinations thereof as shown in Table 3 and Table 4 as targets.

25. The use of claim 24 for diagnostic applications.

26. The use of claim 24 for therapeutic applications.

27. The use of claim 24 for a screening procedure to identify novel drugs.
